(19) United States
(12) Patent Application Publication
(10) Pub. No.: US 2026/0063512 A1
(43) Pub. Date: Mar. 5, 2026

(54) DIAGNOSTIC FOR OXYGEN SENSORS IN DUAL EXHAUST PATHS

(71) Applicant: Ford Global Technologies, LLC, Dearborn, MI (US)

(72) Inventors: Rani Kiwan, West Bloomfield, MI (US); Chris Paul Glugla, Macomb, MI (US)

(21) Appl. No.: 18/822,757

(22) Filed: Sep. 3, 2024

(51) Int. Cl. G01M 15/10; G01N 33/00

(52) U.S. Cl. CPC G01M 15/104; G01N 33/0006

(57) ABSTRACT

Systems and methods for diagnosing operation of four oxygen sensors of an engine that includes crossover pipes are described. The four oxygen sensors may include a left cylinder bank upstream oxygen sensor, a right cylinder bank upstream oxygen sensor, a left cylinder bank downstream oxygen sensor, and a right cylinder bank downstream oxygen sensor.

### FIELD

The present description relates to a system and methods for diagnosing operation of oxygen sensors for an internal combustion engine. The methods and systems may be beneficial for exhaust systems that include flow direction control valves.

### BACKGROUND AND SUMMARY

An engine may include two cylinder banks and two oxygen sensors per bank of cylinders. The two of the four oxygen sensors may be referred to as upstream oxygen sensors since these two oxygen sensors are each positioned upstream of a catalyst. The other two of the four oxygen sensors may be referred to as downstream oxygen sensors since these two oxygen sensors are each positioned downstream of the catalyst. Each of the upstream oxygen sensors may supply feedback to an inner fuel control loop (one inner loop for each cylinder bank) and each of the downstream oxygen sensors may supply feedback to an outer fuel control loop (one outer loop for each cylinder bank). The inner loop fuel controller for a cylinder bank generates larger corrections for engine air-fuel errors and the outer loop fuel controller for the cylinder bank generates smaller corrections in the engine's air-fuel ratio to maintain an oxygen and reductant balance in the cylinder bank's catalyst. A large correction value being generated by the outer loop fuel controller of a cylinder bank may be indicative of an upstream or downstream oxygen sensor degradation. However, the large correction value may be insufficient to identify which of the upstream and downstream oxygen sensors may be degraded.

The inventors herein have recognized the above-mentioned disadvantages and have developed an oxygen sensor diagnostic method, comprising: via a controller, adjusting a first valve configured to adjust flow of exhaust through a first crossover pipe; and identifying degradation of a sole oxygen sensor in response to an operating state of the first valve and in response to output of a first outer loop fuel controller correction value.

By diagnosing operation of oxygen sensors based on outer loop fuel controller output and a position of a valve in an exhaust system, it may be possible to identify degradation of individual oxygen sensors in an exhaust system that includes multiple oxygen sensors. For example, if a right cylinder bank upstream oxygen sensor is degraded causing engine air-fuel ratio control errors, output of a right cylinder bank outer loop fuel controller may exceed a threshold value when a valve in a right cylinder bank exhaust system allows exhaust to flow to a right cylinder bank downstream exhaust sensor. On the other hand, if the same right cylinder bank upstream oxygen sensor is degraded, output of a left cylinder bank outer loop fuel controller may exceed a threshold value when the valve in the right cylinder bank exhaust system allows exhaust to flow to a left cylinder bank downstream exhaust sensor.

The present description may provide several advantages. In particular, the approach may allow for identification of degraded individual oxygen sensors in an engine that includes a plurality of oxygen sensors. Additionally, the approach may reduce engine emissions when an oxygen sensor degrades. Further, the approach may obviate replacing two oxygen sensors of a cylinder bank when a sole one of the cylinder bank's oxygen sensors is degraded.

The above advantages and other advantages, and features of the present description will be readily apparent from the following Detailed Description when taken alone or in connection with the accompanying drawings.

It may be understood that the summary above is provided to introduce in simplified form a selection of concepts that are further described in the detailed description. It is not meant to identify key features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The advantages described herein will be more fully understood by reading an example of an embodiment, referred to herein as the Detailed Description, when taken alone or with reference to the drawings, where:

FIG. 1 is a schematic diagram of a single cylinder of an engine;

FIG. 2 is a schematic diagram of an eight-cylinder engine with valves arranged in a first configuration in an exhaust system to provide straight exhaust flow through a catalyst;

FIG. 3 is a schematic diagram of an eight-cylinder engine with valves arranged in a first configuration in an exhaust system to provide crossover exhaust flow through a catalyst;

FIG. 4 is a schematic diagram of an eight-cylinder engine with valves arranged in a second configuration in an exhaust system to provide straight exhaust flow through a catalyst;

FIG. 5 is a schematic diagram of an eight-cylinder engine with valves arranged in a second configuration in an exhaust system to provide crossover exhaust flow through a catalyst;

FIG. 6 shows an example closed loop fuel control system that includes an inner fuel control loop and an outer fuel control loop for each cylinder bank; and

FIG. 7 shows an example method for diagnosing oxygen sensors of an internal combustion engine.

### DETAILED DESCRIPTION

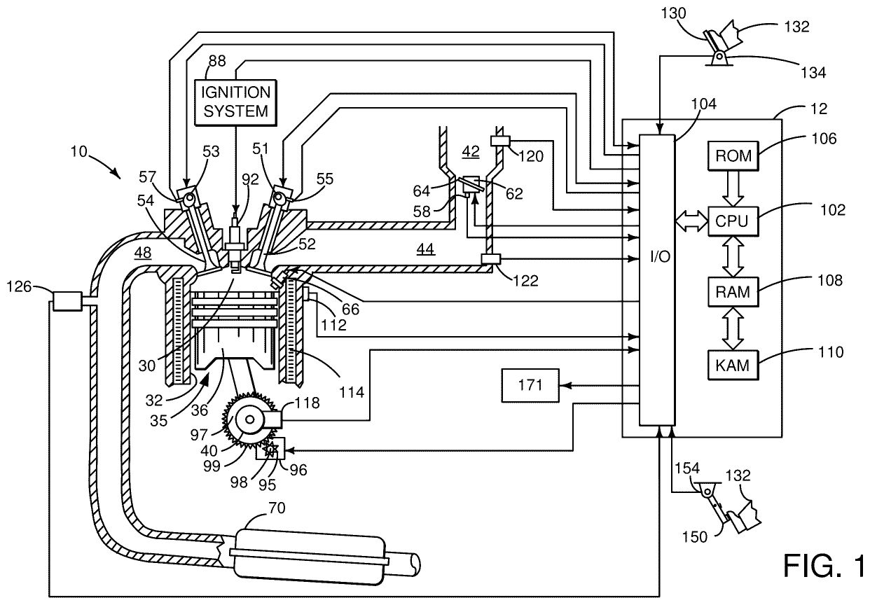

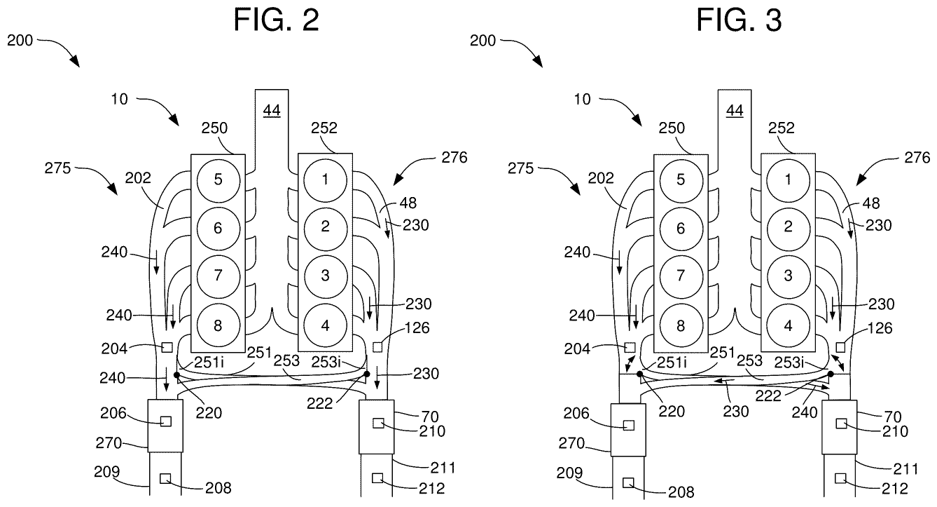

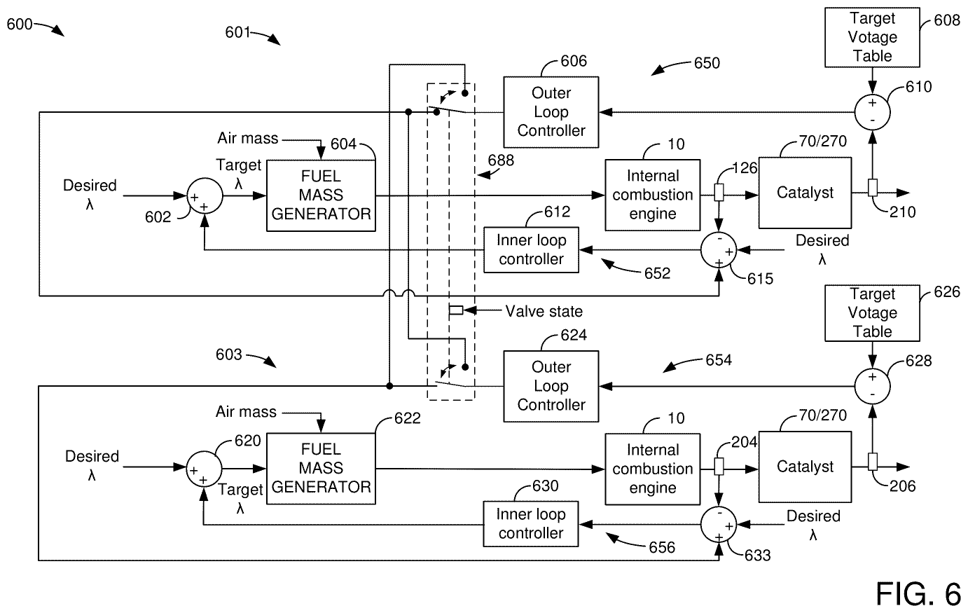

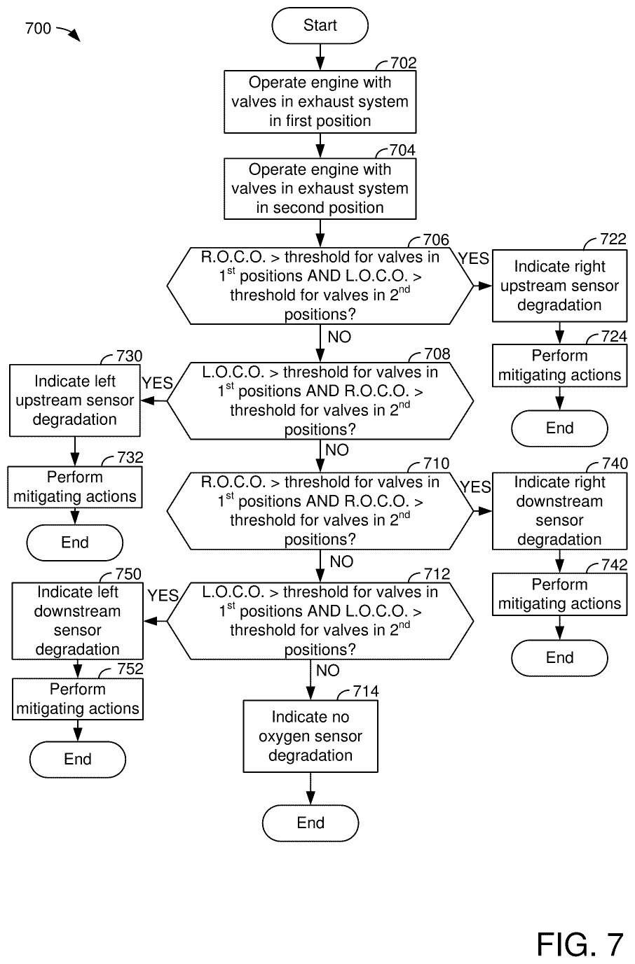

The present description is related to diagnosing individual oxygen sensors for degradation. The oxygen sensors sense exhaust gases from two cylinder banks. An exhaust system is coupled to the two cylinder banks and the exhaust system includes two valves that allow exhaust to flow in different directions. In a first direction, exhaust flows via a short passage to reduce catalyst light-off time. In a second direction, exhaust flows via a longer passage to lower exhaust system temperatures and support high engine speeds and loads. The fuel may be controlled for an internal combustion engine of the type that is shown in FIG. 1. The engine's exhaust system and valves in the exhaust system may be configured with crossover pipes as shown in FIGS. 2-5. The fuel may be controlled via a control system as shown in FIG. 6. Finally, a method for diagnosing oxygen sensors is shown in FIG. 7.

Referring to FIG. 1, internal combustion engine 10, comprising a plurality of cylinders, one cylinder of which is shown in FIG. 1, is controlled by electronic engine controller 12. Engine 10 includes combustion chamber 30 and cylinder walls 32 with piston 36 positioned therein and connected to crankshaft 40. Flywheel 97 and ring gear 99 are coupled to crankshaft 40. Starter 96 includes pinion shaft 98 and pinion gear 95. Pinion shaft 98 may selectively advance pinion gear 95 to engage ring gear 99. Starter 96 may be directly mounted to the front of the engine or the rear of the engine. In one example, starter 96 is in a base state when not engaged to the engine crankshaft. Combustion chamber 30 is shown communicating with intake manifold 44 and exhaust manifold 48 via respective intake valve 52 and exhaust valve 54. Each intake and exhaust valve may be operated by an intake cam 51 and an exhaust cam 53. The position of intake cam 51 may be determined by intake cam sensor 55. The position of exhaust cam 53 may be determined by exhaust cam sensor 57.

Direct fuel injector 66 is shown positioned to inject fuel directly into cylinder 35, which is known to those skilled in the art as direct injection. Fuel injector 66 delivers liquid fuel in proportion to a voltage pulse width or fuel injector pulse width of a signal from controller 12. Fuel is delivered to fuel injector 66 by a fuel system (not shown) including a fuel tank, fuel pump, and fuel rail (not shown). In addition, intake manifold 44 is shown communicating with optional electronic throttle 62 which adjusts a position of throttle plate 64 to control air flow from air intake 42 to intake manifold 44.

Distributorless ignition system 88 provides an ignition spark to combustion chamber 30 via spark plug 92 in response to controller 12. Right cylinder bank upstream oxygen sensor 126 (e.g., universal Exhaust Gas Oxygen (UEGO) sensor, which may be referred to as a wide-band oxygen sensor) is shown coupled to exhaust manifold 48 upstream of catalytic converter 70. Alternatively, a two-state (e.g., narrow band) exhaust gas oxygen sensor may be substituted for right cylinder bank upstream oxygen sensor 126.

Catalytic converter 70 can include multiple catalyst bricks, in one example. In another example, multiple emission control devices, each with multiple bricks, can be used. Catalytic converter 70 can be a three-way type catalyst in one example.

Controller 12 is shown in FIG. 1 as a conventional microcomputer including: microprocessor unit 102, input/output ports 104, read-exclusive memory 106 (e.g., non-transitory memory), random access memory 108, keep alive memory 110, and a conventional data bus. Controller 12 is shown receiving various signals from sensors coupled to engine 10, in addition to those signals previously discussed, including: engine coolant temperature (ECT) from temperature sensor 112 coupled to cooling sleeve 114; a position sensor 134 coupled to a driver demand pedal 130 for sensing a distance displaced by human 132; a position sensor 154 coupled to caliper application pedal 150 for sensing distance displaced by human 132, a measurement of engine manifold pressure (MAP) from pressure sensor 122 coupled to intake manifold 44; an engine position sensor 118 that senses a position of crankshaft 40; a measurement of air mass entering the engine from sensor 120; and a measurement of throttle position from sensor 58. Barometric pressure may also be sensed (sensor not shown) for processing by controller 12. In a preferred aspect of the present description, engine position sensor 118 produces a predetermined number of equally spaced pulses each revolution of the crankshaft from which engine speed (RPM) can be determined.

In some examples, the engine may be coupled to an electric motor/battery system in a hybrid vehicle. Further, in some examples, other engine configurations may be employed, for example a diesel engine with multiple fuel injectors. Further, controller 12 may receive input and communicate conditions such as degradation of components to a light, or alternatively, to human/machine interface 171.

During operation, each cylinder within engine 10 typically undergoes a four stroke cycle: the cycle includes the intake stroke, compression stroke, expansion stroke, and exhaust stroke. During the intake stroke, generally, the exhaust valve 54 closes and intake valve 52 opens. Air is introduced into combustion chamber 30 via intake manifold 44, and piston 36 moves to the bottom of the cylinder so as to increase the volume within combustion chamber 30. The position at which piston 36 is near the bottom of the cylinder and at the end of its stroke (e.g. when combustion chamber 30 is at its largest volume) is typically referred to by those of skill in the art as bottom dead center (BDC). During the compression stroke, intake valve 52 and exhaust valve 54 are closed. Piston 36 moves toward the cylinder head so as to compress the air within combustion chamber 30. The point at which piston 36 is at the end of its stroke and closest to the cylinder head (e.g. when combustion chamber 30 is at its smallest volume) is typically referred to by those of skill in the art as top dead center (TDC). In a process hereinafter referred to as injection, fuel is introduced into the combustion chamber. In a process hereinafter referred to as ignition, the injected fuel is ignited by known ignition means such as spark plug 92, resulting in combustion. During the expansion stroke, the expanding gases push piston 36 back to BDC. Crankshaft 40 converts piston movement into a rotational torque of the rotary shaft. Finally, during the exhaust stroke, the exhaust valve 54 opens to release the combusted air-fuel mixture to exhaust manifold 48 and the piston returns to TDC. Note that the above is shown merely as an example, and that intake and exhaust valve opening and/or closing timings may vary, such as to provide positive or negative valve overlap, late intake valve closing, or various other examples.

Referring now to FIG. 2, a plan view 200 of engine 10 is shown. Engine 10 is the same engine as shown in FIG. 1, but in FIG. 2, all engine cylinders are shown. In this example, the engine's cylinders are numbered 1 through 8. The cylinders are supplied with air via intake manifold 44. A right bank of cylinders includes cylinders 1-4 and a left bank of cylinders includes cylinders 5-8. Cylinders 1-4 are shown in fluidic communication with exhaust manifold 48 and cylinders 5-8 are shown in fluidic communication with exhaust manifold 202. Right cylinder bank exhaust system 276 includes exhaust manifold 48, right cylinder bank upstream oxygen sensor 126, catalyst 70, right cylinder bank downstream oxygen sensor 210, and exhaust pipe 211. Left cylinder bank exhaust system 275 includes exhaust manifold 202, left cylinder bank upstream oxygen sensor 204, catalyst 270, left cylinder bank downstream oxygen sensor 206, and exhaust pipe 209. Each of cylinders 1-8 includes a fuel injector, spark plug, and intake/exhaust valves as shown in FIG. 1.

The engine 10 of FIG. 2 includes catalytic converters 70 and 270 (e.g., close coupled catalysts) that enable fast catalyst light-off when engine 10 is cold started. However, during conditions when the engine is warm and operated at high loads and high speeds for longer periods of time, flowing exhaust gas from left cylinder bank 250 to catalytic converter 270 may cause degradation of catalytic converter 270. In order to reduce a possibility of degrading of catalytic converter 270 during high speed/high load conditions, a position of valve 220 (e.g., left cylinder bank left valve) may be adjusted to direct exhaust to left-to-right crossover pipe 251, which causes exhaust to flow from left cylinder bank 250 to catalytic converter 70. Similarly, in order to reduce a possibility of degrading of catalytic converter 70 during high speed/high load conditions, a position of valve 222 (e.g., a right cylinder bank right valve) may be adjusted to direct exhaust to right-to-left crossover pipe 253, which causes exhaust to flow from right cylinder bank 252 to catalytic converter 270. Left-to-right crossover pipe 251 selectively couples left cylinder bank exhaust system 275 to right cylinder bank exhaust system 276. Similarly, right-to-left crossover pipe 253 selectively couples right cylinder bank exhaust system 276 to left cylinder bank exhaust system 275.

In FIG. 2, a first valve configuration where valve 220 is positioned at inlet 251i of left-to-right crossover pipe 251 and valve 222 is positioned at inlet 253i of right-to-left crossover pipe 253 is shown. Valve 220 is shown in a first position (e.g., a pass through state) where exhaust gas from left cylinder bank 250 bypasses left-to-right crossover pipe 251 and flows a short distance to catalytic converter 270, thereby reducing light-off time of catalytic converter 270 so that engine tailpipe emissions may be reduced. Similarly, valve 222 is shown in a first position where exhaust gas from right cylinder bank bypasses right-to-left crossover pipe 253 and flows a short distance to catalytic converter 70, thereby reducing light-off time of catalytic converter 70 so that engine tailpipe emissions may be reduced. Arrows 240 show a direction of exhaust gas flow from left cylinder bank 250 when valve 220 is blocking exhaust flow from left-to-right crossover pipe 251 as shown. Arrows 230 show a direction of exhaust gas flow from right cylinder bank 252 when valve 222 is blocking exhaust flow from right-to-left crossover pipe 253 as shown.

The right cylinder bank upstream oxygen sensor 126 (e.g., an upstream wide band oxygen sensor (UEGO)) is shown configured to sense exhaust gases from cylinders numbered 1-4 of right cylinder bank 252. The left cylinder bank upstream oxygen sensor 204 (e.g., an upstream wide band oxygen sensor UEGO)) is shown configured to sense exhaust gases from cylinders 5-8 of left cylinder bank 250. A right cylinder bank downstream oxygen sensor 210 (e.g., a downstream narrow band oxygen sensor (HEGO)) is shown configured to sense exhaust gases from within catalytic converter 70, or alternatively, at location 212. A left cylinder bank downstream oxygen sensor 206 (e.g., a downstream narrow band oxygen sensor (HEGO)) is shown configured to sense exhaust gases from within catalytic converter 270, or alternatively, at location 208.

Output of right cylinder bank upstream oxygen sensor 126 may be applied as air-fuel or equivalence ratio (e.g., λ=air-fuel ratio/stoichiometric air-fuel ratio) feedback for controlling fuel that is supplied to cylinders numbered 1-4. Output of left bank cylinder upstream oxygen sensor 204 may be applied as air-fuel or equivalence ratio feedback for controlling fuel that is supplied to cylinders numbered 5-8. Output of right cylinder bank downstream oxygen sensor 210 may be applied as a voltage signal, air-fuel ratio, or equivalence ratio feedback for an outer-loop fuel controller. Output of left cylinder bank downstream oxygen sensor 206 may be applied as air-fuel or equivalence ratio feedback for an outer-loop controller.

Referring now to FIG. 3, the plan view 200 of engine 10 is shown again. The components of engine 10 are the same as shown in FIG. 2 and the components of engine 10 operate as previously described. Therefore, for the sake of brevity the description of engine 10 and its components is omitted for FIG. 3.

In FIG. 3, valve 220 is positioned at inlet 251i of left-to-right crossover pipe 251 and valve 222 is positioned at inlet 253i of right-to-left crossover pipe 253. Valve 220 is shown in a second position (e.g., a bypass state) where exhaust gas from left cylinder bank 250 are blocked from entering catalytic converter 270. Instead, exhaust gases from left cylinder bank 250 are directed to left-to-right crossover pipe 251 and the exhaust flows a longer distance to catalytic converter 70, thereby reducing an amount of heat that may be transferred to catalytic converter 70 so that a possibility of catalyst degradation may be reduced. Similarly, valve 222 is shown in a second position where exhaust gas from right cylinder bank 252 are blocked from entering catalytic converter 70. Rather, exhaust gases from right cylinder bank 252 are directed to right-to-left crossover pipe 253 and exhaust flows a longer distance to catalytic converter 270 so that a possibility of catalyst degradation may be reduced. Arrows 240 show a direction of exhaust gas flow from left cylinder bank 250 when valve 220 is blocking exhaust flow from left cylinder bank 250 to catalytic converter 270 as shown. Arrows 230 show a direction of exhaust gas flow from right cylinder bank 252 when valve 222 is blocking exhaust flow from right cylinder bank 252 to catalytic converter 70. Thus, FIG. 3 shows a configuration where exhaust gases from left cylinder bank are processed via catalytic converter 70 and exhaust gases from right cylinder bank are processed via catalytic converter 270.

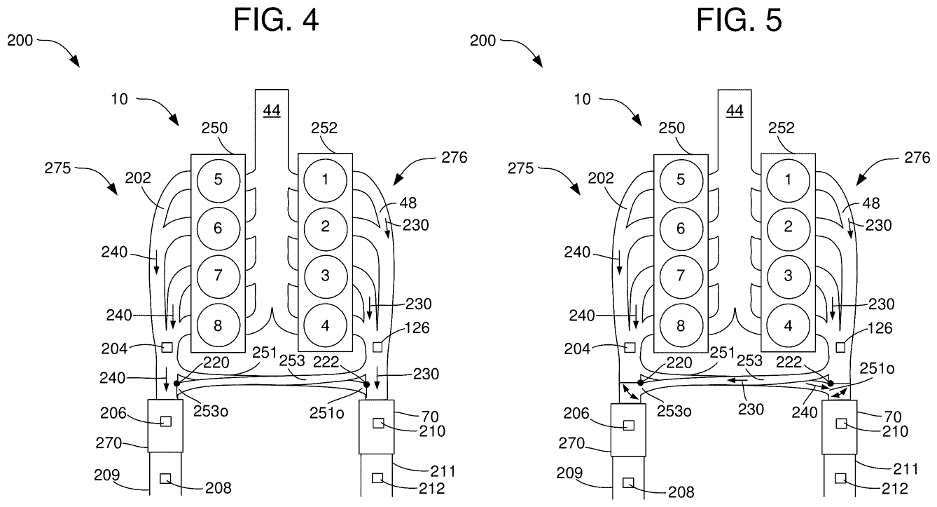

Referring now to FIG. 4, the plan view 200 of engine 10 is shown again. The components of engine 10 are the same as shown in FIG. 2 and the components of engine 10 operate as previously described. Therefore, for the sake of brevity the description of engine 10 and its components is omitted for FIG. 4.

In FIG. 4, a second valve configuration where valve 220 is positioned at outlet 2530 of right-to-left crossover pipe 253 and valve 222 is positioned at outlet 2510 of left-to-right crossover pipe 251 is shown. Valve 220 is shown in a first position where exhaust gas from right cylinder bank 252 bypasses right-to-left crossover pipe 253 and flows a short distance to catalytic converter 70, thereby reducing light-off time of catalytic converter 70 so that engine tailpipe emissions may be reduced. Similarly, valve 222 is shown in a first position where exhaust gas from left cylinder bank 250 bypasses left-to-right crossover pipe 253 and flows a short distance to catalytic converter 270, thereby reducing light-off time of catalytic converter 270 so that engine tailpipe emissions may be reduced. Arrows 240 show a direction of exhaust gas flow from left cylinder bank 250 when valve 222 is blocking exhaust flow from left-to-right crossover pipe 251 as shown. Arrows 230 show a direction of exhaust gas flow from right cylinder bank 252 when valve 220 is blocking exhaust flow from right-to-left crossover pipe 253 as shown.

Referring now to FIG. 5, the plan view 200 of engine 10 is shown yet again. The components of engine 10 are the same as shown in FIG. 2 and the components of engine 10 operate as previously described. Therefore, for the sake of brevity the description of engine 10 and its components is omitted for FIG. 5.

In FIG. 5, the second configuration where valve 220 is positioned at outlet 2530 of right-to-left crossover pipe 253 and valve 222 is positioned at outlet 2510 of left-to-right crossover pipe 251 is shown a second time. Valve 220 is shown in a second position where exhaust gas from left cylinder bank 250 is blocked from catalytic converter 270 and it is permitted to flow through left-to-right crossover pipe 251 and catalytic converter 70. Likewise, valve 222 is shown in a second position where exhaust gas from right cylinder bank 252 is blocked from catalytic converter 70 and it is permitted to flow through right-to-left crossover pipe 253 to catalytic converter 270. The second position of these valves allows catalytic converter 70 and catalytic converter 270 to remain cooler even at high engine speeds and loads. Arrows 240 show a direction of exhaust gas flow from left cylinder bank 250 when valve 220 is blocking exhaust flow from left cylinder bank 250 to catalytic converter 270 as shown. Arrows 230 show a direction of exhaust gas flow from right cylinder bank 252 when valve 222 is blocking exhaust flow from right cylinder bank 252 to catalytic converter 70 as shown.

Thus, the system of FIGS. 1-5 provides for an engine system, comprising: an engine including a left cylinder bank and a right cylinder bank; a right cylinder bank exhaust system coupled to the right cylinder bank; a left cylinder bank exhaust system coupled to the left cylinder bank; a right to left crossover pipe coupling the right cylinder bank exhaust system to the left cylinder bank exhaust system; a left to right crossover pipe coupling the left cylinder bank exhaust system to the right cylinder bank exhaust system; a right valve positioned along the right cylinder bank exhaust system; a left valve positioned along the left cylinder bank exhaust system; a left upstream oxygen sensor; a right upstream oxygen sensor; a left downstream oxygen sensor; a right downstream oxygen sensor; and a controller including executable instructions stored in non-transitory memory that cause the controller to diagnose operation of the left upstream oxygen sensor, the right upstream oxygen sensor, the left downstream oxygen sensor, and the right downstream oxygen sensor via adjusting operating states of the right valve, the left valve, and outputs of two outer loop fuel controllers generated via the controller. In a first example, the engine system includes where a first of the two outer loop fuel controllers generates a first outer loop correction value based on output of the right downstream oxygen sensor. In a second example that may include the first example, the engine system includes where a second of the two outer loop fuel controllers generates a second outer loop correction value based on output of the left downstream oxygen sensor. In a third example that may include one or both of the first and second examples, the engine system further comprises additional executable instructions that cause the controller to perform mitigating actions in response to determining of a degraded oxygen sensor. In a fourth example that may include one or more of the first through third examples, the engine system includes where the mitigating actions include generating a constant value to output from at least one of the two outer loop fuel controllers. In a fifth example that may include one or more of the first through fourth examples, the engine system includes where the mitigating actions include generating a constant value to output from at least one of two inner loop fuel controllers generated via the controller. In a sixth example that may include one or more of the first through fifth examples, the engine system further comprises additional executable instructions that cause the controller to provide an indication of oxygen sensor degradation in response to determining of a degraded oxygen sensor.

Referring now to FIG. 6, a block diagram 600 of a fuel control method and system that includes a right cylinder bank fuel controller 601 and a left cylinder bank fuel controller 603 is shown. The right cylinder bank fuel controller 601 controls the amounts of fuel that are supplied to the right bank of engine cylinders and the left cylinder bank fuel controller 603 controls the amounts of fuel that are supplied to the left bank of engine cylinders. This fuel control system allows outer-loop controller corrections to be switched or swapped between the left cylinder bank fuel controller 603 and the right cylinder bank fuel controller 601 so that outer-loop controller corrections change with positions of left cylinder bank left valve 220 and right cylinder bank right valve 222.

It may be appreciated that FIG. 6 shows a simplified version of a fuel control method and system that includes inner and outer loops for left and right cylinder banks. Other versions of fuel control systems having inner and outer control loops for left and right cylinder banks are also anticipated. At least portions of the fuel control method and system that is shown in FIG. 6 may be generated via executable instructions that are stored in non-transitory memory of a controller (e.g., 12 of FIG. 1).

Right cylinder bank fuel controller 601 includes a right cylinder bank inner control loop 652 and a right cylinder bank outer control loop 650. A desired or requested lambda value (e.g., lambda=air-fuel ratio/stoichiometric air-fuel ratio) for the right cylinder bank is input to right cylinder bank summing junction 602 where it is added with a right cylinder bank inner-loop correction lambda value. Right cylinder bank summing junction 602 outputs a target lambda value for the right cylinder bank that is input to block 604 and block 604 generates a fuel mass for the right cylinder bank 252 in response to the target lambda value for the right cylinder bank and a mass air flow rate into the engine. Block 604 outputs a fuel mass that is delivered to the right cylinder bank 252 of internal combustion engine 10. Internal combustion engine 10 combusts the fuel mass with air to generate torque and exhaust. The exhaust from the right cylinder bank is sensed via right cylinder bank upstream oxygen sensor 126 before the exhaust is processed by catalytic converter 70 or catalytic converter 270 depending on positions of valves 220 and 222.

Right cylinder bank outer control loop 650 receives input from right cylinder bank downstream oxygen sensor 210 and output from right cylinder bank downstream oxygen sensor 210 is subtracted from output of a target voltage table 608 at junction 610. The downstream oxygen sensor voltage error value generated at junction 610 is input to right cylinder bank outer-loop controller 606 (e.g., right cylinder bank outer loop controller) where a first outer-loop correction value is generated. The first outer-loop correction value may be supplied to either summing junction 615, or alternatively, summing junction 633 via switch 688. In this example, control logic is shown as switch 688 which is configured as a double pole double throw switch whose operating state is controlled via positions of or commands to valves in the exhaust (e.g., valve 220 and 222). If valves 220 and 222 are commanded or positioned to allow exhaust from right cylinder bank 252 to flow to catalytic converter 70, the first outer-loop correction value is input to summing junction 615. If valves 220 and 222 are commanded or positioned to allow exhaust from left cylinder bank 250 to flow to catalytic converter 70, the first outer-loop correction value is input to summing junction 633.

Right cylinder bank inner control loop 652 receives input from right cylinder bank upstream oxygen sensor 126 and it is subtracted from the desired lambda value and the output of the right cylinder bank outer control loop or output of the left cylinder bank outer control loop at summing junction 615. Summing junction 615 output is input to right cylinder bank inner-loop controller 612. Right cylinder bank inner-loop controller 612 outputs an inner-loop correction and that output is input to summing junction 602 where it is added to the desired lambda value.

Left cylinder bank fuel controller 603 includes a left cylinder bank inner control loop 656 and a left cylinder bank outer control loop 654. A desired or requested lambda value (e.g., lambda=air-fuel ratio/stoichiometric air-fuel ratio) for the left cylinder bank is input to left cylinder bank summing junction 620 where it is added with a left cylinder bank inner-loop correction lambda value. Left cylinder bank summing junction 620 outputs a target lambda value for the left cylinder bank that is input to block 622 and block 622 generates a fuel mass for the left cylinder bank 250 in response to the target lambda value for the left cylinder bank and a mass air flow rate into the engine. Block 622 outputs a fuel mass that is delivered to the left cylinder bank 250 of internal combustion engine 10. Internal combustion engine 10 combusts the fuel mass with air to generate torque and exhaust. The exhaust from the left cylinder bank is sensed via left cylinder bank upstream oxygen sensor 204 before the exhaust is processed by catalytic converter 70 or catalytic converter 270 depending on positions of valves 220 and 222.

Left cylinder bank outer control loop 654 receives input from left cylinder bank downstream oxygen sensor 206 and output from left cylinder bank downstream oxygen sensor 206 is subtracted from output of a target voltage table 626 at junction 628. The downstream oxygen sensor voltage error value generated at junction 628 is input to left cylinder bank outer-loop controller 624 where a second outer-loop correction value is generated. The second outer-loop correction value may be supplied to either summing junction 633, or alternatively, summing junction 615 via switch 688. If valves 220 and 222 are commanded or positioned to allow exhaust from left cylinder bank 250 to flow to catalytic converter 270, the second outer-loop correction value is input to summing junction 633. If valves 220 and 222 are commanded or positioned to allow exhaust from right cylinder bank 252 to flow to catalytic converter 270, the second outer-loop correction value is input to summing junction 615.

Left cylinder bank inner control loop 656 receives input from left cylinder bank upstream oxygen sensor 204 and it is subtracted from the desired lambda value and the output of the left cylinder bank outer control loop or output of the right cylinder bank outer control loop at summing junction 633. Summing junction 633 output is input to left cylinder bank inner-loop controller 630. Left cylinder bank inner-loop controller 630 outputs an inner-loop correction and that output is input to summing junction 620 where it is added to the desired lambda value.

Thus, as shown and discussed with regard to FIG. 6, outer-loop corrections (Δλ_{c,OL}, e.g., output of block 606) may be applied as a modification of an inner-loop target λ (λ_{t,IL}, e.g., input to block 612) such that: λ_{t,IL}=λ_{des}+Δλ_{c,OL}, where λ_{des} is the desired lambda value. As valves and the exhaust system switch back and forth from direct or straight flow (e.g., a direct exhaust path that does not include flowing through a crossover pipe) to crossover exhaust flow (e.g., exhaust flows from a cylinder bank through a crossover pipe) the outer-loop corrections may be assigned to correct targets of different inner-loops according to the following equations:

For direct exhaust flow paths:

$$\begin{matrix}{λ}_{t , IL} [ R ] = {λ}_{des} + Δ {λ}_{c , O L} [ R ] \\ {λ}_{t , IL} [ L ] = {λ}_{des} + Δ {λ}_{c , O L} [ L ]\end{matrix}$$

For crossover exhaust flow paths:

$$\begin{matrix}{λ}_{t , IL} [ R ] = {λ}_{des} + Δ {λ}_{c , O L} [ L ] \\ {λ}_{t , IL} [ L ] = {λ}_{des} + Δ {λ}_{c , O L} [ R ]\end{matrix}$$

where [R] identifies the right cylinder bank, [L] identifies the left cylinder bank, Δλ_{c,OL} corresponds to the lambda correction for an outer-loop, λ_{des} is desired lambda, λ_{t,IL} is target lambda for the inner-loop controller. During a time when valves in the exhaust are moving, outer loop corrections may be held at their most recent value.

It may be appreciated that instead of switching the output of right cylinder bank outer loop controller 606 from summing junction 615 to summing junction 633 and switching the output of left cylinder bank outer loop controller 624 from summing junction 633 to summing junction 615, output of right cylinder bank downstream oxygen sensor 210 may be switched from summing junction 610 to summing junction 628 and output of left cylinder bank downstream oxygen sensor 206 may be switched from summing junction 628 to summing junction 610 in response to changing the position of valves 220 and 222 to perform substantially a same function and achieve a substantially same result as switching the destination of outputs of the outer loop controllers 606 and 624. Additionally, output of target voltage table 626 may be switched to summing junction 610 and output of target voltage table 608 may be switched to summing junction 628.

Moving on to FIG. 7, a flow chart of a method for diagnosing oxygen sensors of an internal combustion engine is shown. The method of FIG. 7 may be incorporated into the system of FIGS. 1-5 as executable instructions stored in non-transitory memory. The method of FIG. 7 may cause the controller shown in FIG. 1 to receive inputs from one or more sensors described herein and adjust positions or operating states of one or more actuators described herein in the physical world.

At 702, method 700 operates the engine with valves in its exhaust system (e.g., valves 220 and 222) in a first position or pass through state (straight exhaust flow). When the valves are operated in the first position, exhaust flows from the right cylinder bank 252 to the first upstream oxygen sensor, or the right cylinder bank upstream oxygen sensor 126, and then to the right cylinder bank downstream oxygen sensor 210, and from the left cylinder bank 250 to the second upstream oxygen sensor, or the left cylinder bank upstream oxygen sensor 204, and then to the left cylinder bank downstream oxygen sensor 206.

The engine operates by rotating and combusting air and fuel that enters engine cylinders. The air-fuel ratios are controlled via controllers as shown in FIG. 6. The cylinder air-fuel ratios may vary (e.g., cycle rich and lean) about a stoichiometric average mixture as the engine operates. Method 700 monitors and stores to memory the correction values that are output from the outer loop controllers (e.g., the output values from blocks 606 and 624 shown in FIG. 6) after the valves in the exhaust system have been in the first position for a predetermined amount of time. Method 700 proceeds to 704.

At 704, method 700 operates the engine with valves in its exhaust system (e.g., valves 220 and 222) in a second position or bypass state (crossover exhaust flow). When the valves are operated in the second position, exhaust flows from the right cylinder bank 252 to the first upstream oxygen sensor, or the right cylinder bank upstream oxygen sensor 126, and then to the left cylinder bank downstream oxygen sensor 206, and from the left cylinder bank 250 to the second upstream oxygen sensor, or the left cylinder bank upstream oxygen sensor 204, and then to the right cylinder bank downstream oxygen sensor 210.

The engine operates by rotating and combusting air and fuel that enters engine cylinders. The air-fuel ratios are controlled via controllers as shown in FIG. 6. The cylinder air-fuel ratios may vary (e.g., cycle rich and lean) about a stoichiometric average mixture as the engine operates. Method 700 monitors and stores to memory the correction values that are output from the outer loop controllers (e.g., the output values from blocks 606 and 624 shown in FIG. 6) after the valves in the exhaust system have been in the second position for a predetermined amount of time. Method 700 proceeds to 706.

At 706, method 700 judges if the right cylinder bank outer loop controller (e.g., 606 of FIG. 6) is outputting a correction value (right outer-loop correction output (R.O.C.O.)) that is greater than a threshold value when the exhaust valves (e.g., 220 and 222 of FIG. 2) are in their first position (e.g., pass through state (straight exhaust flow)) and if the left cylinder bank outer loop controller (e.g., 624 of FIG. 6) is outputting a correction value (L.O.C.O) that is greater than a threshold value when the exhaust valves (e.g., 220 and 222 of FIG. 2) are in their second position (e.g., bypass state (crossover exhaust flow)). This logic is indicative of the right cylinder bank upstream oxygen sensor (e.g., 126 of FIG. 2) being degraded because the right cylinder bank upstream oxygen sensor is the sole oxygen sensor that has ability to cause both outer loop controller corrections to exceed threshold values during such conditions. Therefore, if these conditions are met, the answer is yes and method 700 proceeds to 722. Otherwise, the answer is no and method 700 proceeds to 708.

At 722, method 700 indicates that the right cylinder bank upstream oxygen sensor is degraded. In one example, method 700 provides an indication that the right cylinder bank upstream oxygen sensor is degraded via a human/machine interface. Method 700 proceeds to 724.

At 724, method 700 performs mitigating actions to reduce effects of a degraded right cylinder bank upstream oxygen sensor. The mitigating actions may include, but are not constrained to operating the right cylinder bank in an inner loop open loop mode where fuel is supplied to the right cylinder bank based on the amount of air entering cylinders of the right cylinder bank without feedback from the right cylinder bank upstream oxygen sensor. The right cylinder bank outer loop fuel controller or the left cylinder bank outer loop fuel controller may adjust the amount of fuel that is supplied to the right cylinder bank. Alternatively, the right cylinder bank may be supplied with fuel according to feedback from the left cylinder bank inner loop fuel controller. Method 700 proceeds to exit.

At 708, method 700 judges if the left cylinder bank outer loop controller (e.g., 624 of FIG. 6) is outputting a correction value that is greater than a threshold value when the exhaust valves (e.g., 220 and 222 of FIG. 2) are in their first position (e.g., pass through state (straight exhaust flow)) and if the right cylinder bank outer loop controller (e.g., 606 of FIG. 6) is outputting a correction value that is greater than a threshold value when the exhaust valves (e.g., 220 and 222 of FIG. 2) are in their second position (e.g., bypass state (crossover exhaust flow)). This logic is indicative of the left cylinder bank upstream oxygen sensor (e.g., 204 of FIG. 2) being degraded because the left cylinder bank upstream oxygen sensor is the sole oxygen sensor that has ability to cause both outer loop controller corrections to exceed threshold values during such conditions. Therefore, if these conditions are met, the answer is yes and method 700 proceeds to 730. Otherwise, the answer is no and method 700 proceeds to 710.

At 730, method 700 indicates that the left cylinder bank upstream oxygen sensor is degraded. In one example, method 700 provides an indication that the left cylinder bank upstream oxygen sensor is degraded via a human/machine interface. Method 700 proceeds to 732.

At 732, method 700 performs mitigating actions to reduce effects of a degraded left cylinder bank upstream oxygen sensor. The mitigating actions may include, but are not constrained to operating the left cylinder bank in an inner loop open loop mode where fuel is supplied to the left cylinder bank based on the amount of air entering cylinders of the left cylinder bank without feedback from the left cylinder bank upstream oxygen sensor. The left cylinder bank outer loop fuel controller or the right cylinder bank outer loop fuel controller may adjust the amount of fuel that is supplied to the left cylinder bank. Alternatively, the left cylinder bank may be supplied with fuel according to feedback from the right cylinder bank inner loop fuel controller. Method 700 proceeds to exit.

At 710, method 700 judges if the right cylinder bank outer loop controller (e.g., 606 of FIG. 6) is outputting a correction value that is greater than a threshold value when the exhaust valves (e.g., 220 and 222 of FIG. 2) are in their first position (e.g., pass through state) and if the right cylinder bank outer loop controller (e.g., 606 of FIG. 6) is outputting a correction value that is greater than a threshold value when the exhaust valves (e.g., 220 and 222 of FIG. 2) are in their second position (e.g., bypass state). This logic is indicative of the right cylinder bank downstream oxygen sensor (e.g., 210 of FIG. 2) being degraded because the right cylinder bank downstream oxygen sensor is the sole oxygen sensor that has ability to cause both right cylinder bank outer loop controller corrections to exceed threshold values for both exhaust valve positions. Therefore, if these conditions are met, the answer is yes and method 700 proceeds to 740. Otherwise, the answer is no and method 700 proceeds to 712.

At 740, method 700 indicates that the right cylinder bank downstream oxygen sensor is degraded. In one example, method 700 provides an indication that the right cylinder bank downstream oxygen sensor is degraded via a human/machine interface. Method 700 proceeds to 742.

At 742, method 700 performs mitigating actions to reduce effects of a degraded right cylinder bank downstream oxygen sensor. The mitigating actions may include, but are not constrained to operating the right cylinder bank in an outer loop open loop mode while the exhaust valves are in the first position where fuel is supplied to the right cylinder bank based on a constant predetermined output (e.g., zero) for the right cylinder bank outer loop controller, and operating the left cylinder bank in an outer loop open loop mode while the exhaust valves are in the second position where fuel is supplied to the left cylinder bank based on a constant predetermined output (e.g., zero) for the right cylinder bank outer loop controller. Alternatively, the right cylinder bank outer loop controller may output a correction that is equivalent to output of the left cylinder bank outer loop fuel controller. Method 700 proceeds to exit.

At 712, method 700 judges if the left cylinder bank outer loop controller (e.g., 624 of FIG. 6) is outputting a correction value that is greater than a threshold value when the exhaust valves (e.g., 220 and 222 of FIG. 2) are in their first position (e.g., pass through state) and if the left cylinder bank outer loop controller (e.g., 624 of FIG. 6) is outputting a correction value that is greater than a threshold value when the exhaust valves (e.g., 220 and 222 of FIG. 2) are in their second position (e.g., bypass state). This logic is indicative of the left cylinder bank downstream oxygen sensor (e.g., 206 of FIG. 2) being degraded because the left cylinder bank downstream oxygen sensor is the sole oxygen sensor that has ability to cause both left cylinder bank outer loop controller corrections to exceed threshold values for both exhaust valve positions. Therefore, if these conditions are met, the answer is yes and method 700 proceeds to 750. Otherwise, the answer is no and method 700 proceeds to 714.

At 750, method 700 indicates that the left cylinder bank downstream oxygen sensor is degraded. In one example, method 700 provides an indication that the left cylinder bank downstream oxygen sensor is degraded via a human/machine interface. Method 700 proceeds to 752.

At 752, method 700 performs mitigating actions to reduce effects of a degraded left cylinder bank downstream oxygen sensor. The mitigating actions may include, but are not constrained to operating the left cylinder bank in an outer loop open loop mode while the exhaust valves are in the first position where fuel is supplied to the left cylinder bank based on a constant predetermined output (e.g., zero) for the left cylinder bank outer loop controller, and operating the right cylinder bank in an outer loop open loop mode while the exhaust valves are in the second position where fuel is supplied to the right cylinder bank based on a constant predetermined output (e.g., zero) for the left cylinder bank outer loop controller. Alternatively, the left cylinder bank outer loop controller may output a correction that is equivalent to output of the right cylinder bank outer loop fuel controller. Method 700 proceeds to exit.

At 714, method 700 indicates no oxygen sensor degradation and exits. No oxygen sensor degradation may be indicated via a human/machine interface.

Thus, method 700 evaluates the operating status of four oxygen sensors of two cylinder banks individually according to outputs of downstream controllers and positions of valves in an exhaust system. These two parameters enable method 700 to perform diagnostic evaluations for each oxygen sensor individually.

The methods of FIG. 7 provides for an oxygen sensor diagnostic method, comprising: via a controller, adjusting a first valve configured to adjust flow of exhaust through a first crossover pipe; and identifying degradation of a sole oxygen sensor in response to an operating state of the first valve and in response to output of a first outer loop fuel controller correction value. In a first example, the oxygen sensor diagnostic method further comprises adjusting a second valve configured to adjust flow of exhaust through a second crossover pipe, and identifying degradation of the sole oxygen sensor in response to operating states of the second valve and output of a second outer loop fuel controller correction value. In a second example that may include the first example, the oxygen sensor diagnostic method includes where sole oxygen sensor is a right cylinder bank upstream oxygen sensor, and where the operating state of the first valve and the operating state of the second valve are adjusted to diagnose the right cylinder bank upstream oxygen sensor. In a third example that may include one or both of the first and second examples, the oxygen sensor diagnostic method includes where sole oxygen sensor is a left cylinder bank upstream oxygen sensor, and where the operating state of the first valve and the operating state of the second valve are adjusted to diagnose the left cylinder bank upstream oxygen sensor. In a fourth example that includes one or more of the first through third examples, the oxygen sensor diagnostic method includes where sole oxygen sensor is a right cylinder bank downstream oxygen sensor, and where the operating state of the first valve and the operating state of the second valve are adjusted to diagnose the right cylinder bank downstream oxygen sensor. In a fifth example that includes one or more of the first through fourth examples, the oxygen sensor diagnostic method includes where sole oxygen sensor is a left cylinder bank downstream oxygen sensor, and where the operating state of the first valve and the operating state of the second valve are adjusted to diagnose the left cylinder bank downstream oxygen sensor. In a sixth example that includes one or more of the first through fifth examples, the oxygen sensor diagnostic method includes where the first crossover pipe couples an exhaust pipe of a right cylinder bank to an exhaust pipe of a left cylinder bank. In a seventh example that includes one or more of the first through sixth examples, the oxygen sensor diagnostic method includes where the first outer loop fuel controller correction value is based on output of a downstream oxygen sensor of a right cylinder bank.

The methods of FIG. 7 provides for an oxygen sensor diagnostic method, comprising: via a controller, identifying a presence or absence of oxygen sensor degradation for four oxygen sensors according to outputs of two outer loop fuel controllers and operating states of a first valve and a second valve. In a first example, the oxygen sensor diagnostic method includes where the first valve is configured to control exhaust flow through a first crossover pipe that couples a left cylinder bank exhaust system to a right cylinder bank exhaust system. In a second example that may include the first example, the oxygen sensor diagnostic method includes where the second valve is configured to control exhaust flow through a second crossover pipe that couples the left cylinder bank exhaust system to the right cylinder bank exhaust system. In a third example that may include one or both of the first and second examples, the oxygen sensor diagnostic method includes where the first outer loop fuel controller adjusts fuel supplied to a right bank of cylinders. In a fourth example that may include one or more of the first through third examples, the oxygen sensor diagnostic method includes where the second outer loop fuel controller adjusts fuel supplied to a left bank of cylinders.

Note that the example control and estimation routines included herein can be used with various engine and/or vehicle system configurations. In addition, although the methods included herein refer to lambda control, the approaches herein may be applied with other units. For example, the approaches herein describe lambda control, but in other examples, the controls and methods may be configured for air-fuel ratio control. The control methods and routines disclosed herein may be stored as executable instructions in non-transitory memory and may be carried out by the control system including the controller in combination with the various sensors, actuators, and other engine hardware. The specific routines described herein may represent one or more of any number of processing strategies such as event-driven, interrupt-driven, multi-tasking, multi-threading, and the like. As such, various actions, operations, and/or functions illustrated may be performed in the sequence illustrated, in parallel, or in some cases omitted. Likewise, the order of processing is not necessarily required to achieve the features and advantages of the example examples described herein, but is provided for ease of illustration and description. One or more of the illustrated actions, operations and/or functions may be repeatedly performed depending on the particular strategy being used. Further, the described actions, operations and/or functions may graphically represent code to be programmed into non-transitory memory of the computer readable storage medium in the engine control system, where the described actions are carried out by executing the instructions in a system including the various engine hardware components in combination with the electronic controller

This concludes the description. The reading of it by those skilled in the art would bring to mind many alterations and modifications without departing from the spirit and the scope of the description. For example, V6, V8, V10, and V12 engines operating in natural gas, gasoline, diesel, or alternative fuel configurations could use the present description to advantage.

1. An oxygen sensor diagnostic method, comprising:
via a controller, adjusting a first valve configured to adjust flow of exhaust through a first crossover pipe; and
identifying degradation of a sole oxygen sensor in response to an operating state of the first valve and in response to output of a first outer loop fuel controller correction value.

2. The oxygen sensor diagnostic method of claim 1, further comprising adjusting a second valve configured to adjust flow of exhaust through a second crossover pipe, and identifying degradation of the sole oxygen sensor in response to operating states of the second valve and output of a second outer loop fuel controller correction value.

3. The oxygen sensor diagnostic method of claim 2, where sole oxygen sensor is a right cylinder bank upstream oxygen sensor, and where the operating state of the first valve and the operating state of the second valve are adjusted to diagnose the right cylinder bank upstream oxygen sensor.

4. The oxygen sensor diagnostic method of claim 2, where sole oxygen sensor is a left cylinder bank upstream oxygen sensor, and where the operating state of the first valve and the operating state of the second valve are adjusted to diagnose the left cylinder bank upstream oxygen sensor.

5. The oxygen sensor diagnostic method of claim 2, where sole oxygen sensor is a right cylinder bank downstream oxygen sensor, and where the operating state of the first valve and the operating state of the second valve are adjusted to diagnose the right cylinder bank downstream oxygen sensor.

6. The oxygen sensor diagnostic method of claim 2, where sole oxygen sensor is a left cylinder bank downstream oxygen sensor, and where the operating state of the first valve and the operating state of the second valve are adjusted to diagnose the left cylinder bank downstream oxygen sensor.

7. The oxygen sensor diagnostic method of claim 1, where the first crossover pipe couples a right cylinder bank exhaust pipe to a left cylinder bank exhaust pipe.

8. The oxygen sensor diagnostic method of claim 1, where the first outer loop fuel controller correction value is based on output of a downstream oxygen sensor of a right cylinder bank.

9. An engine system, comprising:
an engine including a left cylinder bank and a right cylinder bank;
a right cylinder bank exhaust system coupled to the right cylinder bank;
a left cylinder bank exhaust system coupled to the left cylinder bank;
a right to left crossover pipe coupling the right cylinder bank exhaust system to the left cylinder bank exhaust system;
a left to right crossover pipe coupling the left cylinder bank exhaust system to the right cylinder bank exhaust system;
a right valve positioned along the right cylinder bank exhaust system;
a left valve positioned along the left cylinder bank exhaust system;
a left upstream oxygen sensor;
a right upstream oxygen sensor;
a left downstream oxygen sensor;
a right downstream oxygen sensor; and
a controller including executable instructions stored in non-transitory memory that cause the controller to diagnose operation of the left upstream oxygen sensor, the right upstream oxygen sensor, the left downstream oxygen sensor, and the right downstream oxygen sensor via adjusting operating states of the right valve, the left valve, and outputs of two outer loop fuel controllers generated via the controller.

10. The engine system of claim 9, where a first of the two outer loop fuel controllers generates a first outer loop correction value based on output of the right downstream oxygen sensor.

11. The engine system of claim 10, where a second of the two outer loop fuel controllers generates a second outer loop correction value based on output of the left downstream oxygen sensor.

12. The engine system of claim 9, further comprising additional executable instructions that cause the controller to perform mitigating actions in response to determining of a degraded oxygen sensor.

13. The engine system of claim 12, where the mitigating actions include generating a constant value to output from at least one of the two outer loop fuel controllers.

14. The engine system of claim 12, where the mitigating actions include generating a constant value to output from at least one of two inner loop fuel controllers generated via the controller.

15. The engine system of claim 9, further comprising additional executable instructions that cause the controller to provide an indication of oxygen sensor degradation in response to determining of a degraded oxygen sensor.

16. An oxygen sensor diagnostic method, comprising:
via a controller, identifying a presence or absence of oxygen sensor degradation for four oxygen sensors according to outputs of two outer loop fuel controllers and operating states of a first valve and a second valve.

17. The oxygen sensor diagnostic method of claim 16, where the first valve is configured to control exhaust flow through a first crossover pipe that couples a left cylinder bank exhaust system to a right cylinder bank exhaust system.

18. The oxygen sensor diagnostic method of claim 17, where the second valve is configured to control exhaust flow through a second crossover pipe that couples the left cylinder bank exhaust system to the right cylinder bank exhaust system.

19. The oxygen sensor diagnostic method of claim 16, where a first outer loop fuel controller of the two outer loop fuel controllers adjusts fuel supplied to a right bank of cylinders.

20. The oxygen sensor diagnostic method of claim 19, where a second outer loop fuel controller of the two outer loop fuel controllers adjusts fuel supplied to a left bank of cylinders.
